(19) United States
(12) Patent Application Publication
(10) Pub. No.: US 2021/0298586 A1
(43) Pub. Date: Sep. 30, 2021

(54) Cleaning Device for Use with an Endoscope

(71) Applicant: BOSTON SCIENTIFIC SCIMED, INC., Maple Grove, MN (US)

(72) Inventors: ALYSON BOLICK, Boston, MA (US); MATTHEW B. HOLLYER, Watertown, MA (US); KYLE P. MOORE, Alpharetta, GA (US); BARRY WEITZNER, Acton, MA (US)

(73) Assignee: BOSTON SCIENTIFIC SCIMED, INC., Maple Grove, MN (US)

(21) Appl. No.: 17/217,136

(22) Filed: Mar. 30, 2021

Related U.S. Application Data
(60) Provisional application No. 63/002,769, filed on Mar. 31, 2020.

(51) Int. Cl. A61B 1/12; A46B 13/02; A46B 15/00

(52) U.S. Cl. CPC A61B 1/122; A46B 2200/3073; A46B 15/0004; A46B 13/02

(57) ABSTRACT

Apparatuses for cleaning an endoscope as well as methods for making and using the same are disclosed. An example apparatus may include a control unit. A shaft may be configured to be coupled to the control unit. The shaft may have a distal end region. A cleaning member may be coupled to/couplable to the distal end region of the shaft. The control unit may be configured to move the cleaning member.

### CROSS REFERENCE TO RELATED APPLICATIONS

The present application claims the benefit of and priority to U.S. Provisional Patent Application Ser. No. 63/002,769, filed on Mar. 31, 2020, the disclosure of which is incorporated herein by reference.

### TECHNICAL FIELD

The present disclosure pertains to medical devices, and methods for manufacturing medical devices. More particularly, the present disclosure pertains to cleaning devices for use with an endoscope.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An apparatus for cleaning an endoscope is disclosed. The apparatus comprises: a control unit; a shaft configured to be coupled to the control unit, the shaft having a distal end region; a cleaning member coupled to the distal end region of the shaft; and wherein the control unit is configured to move the cleaning member.

Alternatively or additionally to any of the embodiments above, the control unit includes a motor.

Alternatively or additionally to any of the embodiments above, the motor is a direct current motor.

Alternatively or additionally to any of the embodiments above, the control unit is configured to rotate the cleaning member.

Alternatively or additionally to any of the embodiments above, the control unit is configured to translate the cleaning member.

Alternatively or additionally to any of the embodiments above, the control unit is configured to reciprocate the cleaning member.

Alternatively or additionally to any of the embodiments above, the shaft includes a catheter shaft.

Alternatively or additionally to any of the embodiments above, the cleaning member includes one or more bristles.

Alternatively or additionally to any of the embodiments above, the control unit includes a coupling region configured to be coupled to a biopsy port of the endoscope.

Alternatively or additionally to any of the embodiments above, the control unit includes a flush port.

An apparatus for cleaning an endoscope is disclosed. The apparatus comprises: an oscillating motor; a single-use brush assembly coupled to the oscillating motor, the single-use brush assembly including a shaft having a brush coupled thereto; and wherein the oscillating motor is configured to move the single-use brush assembly such that the single-use brush assembly can clean a channel of the endoscope.

Alternatively or additionally to any of the embodiments above, the oscillating motor includes a direct current power supply.

Alternatively or additionally to any of the embodiments above, the oscillating motor is configured to rotate the single-use brush assembly.

Alternatively or additionally to any of the embodiments above, the oscillating motor is configured to translate the single-use brush assembly.

Alternatively or additionally to any of the embodiments above, the oscillating motor is configured to reciprocate the single-use brush assembly.

Alternatively or additionally to any of the embodiments above, the oscillating motor includes housing having a coupling member for coupling the oscillating motor to a biopsy port.

Alternatively or additionally to any of the embodiments above, the oscillating motor includes housing having a flush port.

A method for cleaning a channel of an endoscope is disclosed. The method comprises: coupling a cleaning apparatus to a biopsy port, the cleaning apparatus including a control unit and a brush assembly coupled to the control unit; wherein the brush assembly includes a shaft and a brush coupled to the shaft; disposing the brush with the channel of the endoscope; and actuating the control unit to cause the brush to move within the channel.

Alternatively or additionally to any of the embodiments above, the control unit includes a coupling member and wherein coupling a cleaning apparatus to a biopsy port includes securing the coupling member to the biopsy port.

Alternatively or additionally to any of the embodiments above, actuating the control unit to cause the brush to move within the channel includes translating the brush within the channel, rotating the brush within the channel, or both.

An apparatus for cleaning an endoscope is disclosed. The apparatus comprises: a control unit; a shaft couplable to the control unit, the shaft having a distal end region; a cleaning member couplable to the distal end region of the shaft; and wherein the control unit is configured to move the cleaning member.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates an example endoscope.

FIG. 2 illustrates an example cleaning assembly.

FIG. 3 depicts an example use of a cleaning assembly with an endoscope.

FIG. 4 depicts an example control unit.

FIG. 5 depicts an example use of a cleaning assembly with an endoscope.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

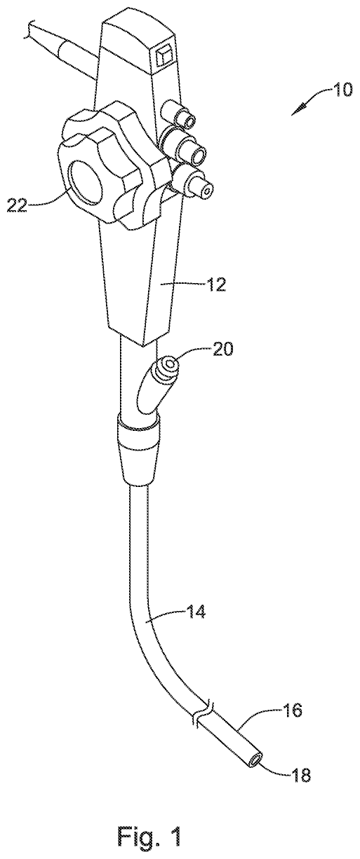

An example endoscope and/or endoscope assembly 10 is illustrated in FIG. 1. The endoscope 10 may be any of a number of types of endoscopes or related medical devices usually identified by the particular anatomy desired to be reached. For example, the endoscope 10 may be a bronchoscope, colonoscope, duodenoscope, esophagoscope, guidetubes, introducers (without or without vision or visualization capabilities), or any other type of endoscope or related medical device. The endoscope 10 may include a handpiece 12 and an elongate shaft 14 extending distally from the handpiece 12 to a distal tip region 16. The handpiece 12, for example, may include a number of controls such as one or more control knobs 22. The shaft 14 may include a lumen defining a working channel 18 extending through the shaft 14. Although the endoscope 10 is depicted with a single working channel in FIG. 1, it can be appreciated that in other embodiments, the endoscope 10 may include multiple working channels, as desired. A biopsy port 20 may be disposed adjacent to the shaft 14 and the biopsy port 20 may provide access to the working channel 18.

Endoscopes like the endoscope 10 are useful medical devices for a number of different medical interventions. Due at least in part to the complex nature and expense associated with endoscopes, standard clinical practices include the cleaning/reprocessing of the endoscope so that the endoscope can be properly prepared for re-use. One part of a typical cleaning process may include the removal of debris from one or more channels formed in the endoscope. Disclosed herein are apparatuses/devices generally designed for use in cleaning/reprocessing endoscopes and the like.

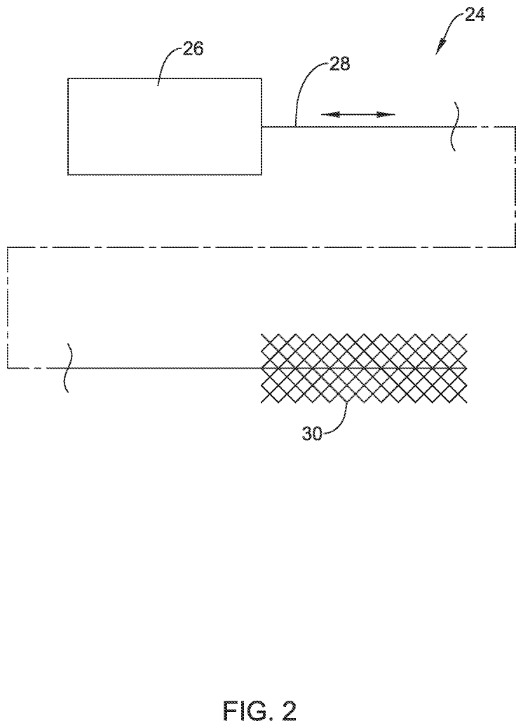

FIG. 2 is a schematic illustration depicting a cleaning assembly 24 that, for example, may be used to clean the endoscope 10. The cleaning assembly 24 may include a control unit 26. A shaft 28 may be coupled to the control unit 26. A cleaning member 30 may be coupled to the shaft 28. In at least some instances, the cleaning member 30 takes the form of a brush, for example having a plurality of bristles, that can be used to scrub and/or otherwise remove debris from the endoscope 10. In such instances, the shaft 28 and the cleaning member 30 may also be termed or otherwise considered to be a brush assembly. The control unit 26 can be used to move the shaft 28 and the cleaning member 30. Thus, as opposed to a manual cleaning process, the control unit 26 can be used to mechanically increase the number of interactions between the cleaning member/brush 30 and the surface in need of cleaning by moving, rotating, translating, vibrating, reciprocating, oscillating, etc. the cleaning member 30.

The control unit 26 may take on a variety of different forms. For example, the control unit 26 may generally include a housing configured to be coupled to the shaft 28. For example, the control unit 26 may include an internal chamber or socket designed to accept and end region of the shaft 28. In some instances, the shaft 28 can be releasably secured to the control unit 26. In some of these and in other instances, the shaft 28 may be coupled to the control unit 26 in a manner that permits using the control unit 26 to move the shaft 28. In some instances, the shaft 28 is coupled to an end region of the control unit 26. In such instances, the shaft 28 may not extend into an internal region of the control unit 26. In other instances, the shaft 28 may extend partially into the control unit 26. In still other instances, the shaft 28 may extend through the control unit 26 (e.g., so that one portion of the shaft 28 extends from one side of the control unit 26 and another portion of the shaft 28 extend from another side of the control unit 26).

The control unit 26 may include a mechanism for moving the shaft 28. In general, the mechanism for moving the shaft 28 may result in axial and/or translation movement, multi-axial movement, circular movement, rotational movement, orbital movement, vibrational movement (e.g., vibration), reciprocating movement (e.g., reciprocation), oscillating movement (e.g., oscillation), and/or the like. In some instances, the mechanism may be a relatively simple wind-up mechanism that, when activated, can vibrate and/or rotate the shaft 28. For example, the control unit 26 may include a potential energy storage device (e.g., a gear train, spring, combinations thereof, etc.). Once the potential energy storage device is put into a state of high potential energy, the potential energy is converted into kinetic energy by releasing the potential energy, and that is translated to the cleaning assembly 24 (e.g., the shaft 28 and/or the cleaning member 30) through vibrations, oscillations, and/or other periodic motions. For example, the potential energy device may include a winding mechanism, a spring, and one more gears coupled to spring. Winding the winding mechanism winds the spring into a more tightly wound configuration. Releasing the winding mechanism may result in the spring “releasing” or unwinding, thereby causing the gears and, ultimately, the shaft 28 to vibrate, rotate, or otherwise move.

In some instances, other mechanisms for moving the shaft 28 are contemplated. For example, mechanisms are contemplated that utilize planetary gear boxes to drive motion of the shaft. Other mechanisms are contemplated that utilize a push-pull helix drill drive, a hand drive similar to that used in an egg beater. These are just examples. Other mechanisms are contemplated.

Engaging the shaft 28 with the mechanism for moving the shaft 28 may include any one of variety of connections and/or connectors. For example, a bayonet connector may be used, a screw/threaded connector may be used, a magnetic coupling may be used. These are just examples. Other connections are contemplated.

In some instances, the control unit 26 may include a motor (e.g., an electric motor). The motor may be coupled to the shaft 28 so as to move, rotate, translate, vibrate, reciprocate, oscillate, etc. the shaft 28 (and, for example, the cleaning member 30). Thus, the motor may be considered to be a rotary motor, vibrating motor, oscillating motor, etc. In some instances, the motor and/or the control unit 26 may utilize and/or otherwise include a power source. In some instances, the control unit 26 may utilize a direct current power source (e.g., battery powered) and/or a direct current motor (e.g., a battery powered motor). In some of these and in other instances, the control unit 26 may include an alternating current power source (e.g., and/or an alternating current motor) and/or another power source (e.g., solar power; e.g. a solar powered motor). In some instances, the control unit 26 may include a piezo electric source of motion where an electric current is converted into motion (e.g., rotation, translation, vibration, oscillation, etc.) by expansion and contraction of the piezo element. In general, the power source may be used to help provide motion to the shaft 28. In some instances, however, a power source may not be needed in order to provide motion to the shaft 28. In some of these and in other instances, a hand crank mechanism may be used to effect motion. In some of these and in other instances, a hand-operated screw type mechanism may be utilized where a user pushes and/or pulls the mechanism to effect motion.

In at least some instances, the control unit 26 may be designed for multiple uses. For example, the control unit 26 can be releasably attached to the shaft 28 (e.g., a shaft 28 with a cleaning member/brush 30 coupled thereto), used in conjunction with the shaft 28 and cleaning member/brush 30 to clean an endoscope 10, detached from the shaft 28, and then re-attached to another shaft 28 (e.g., another shaft 28 with a cleaning member/brush 30 coupled thereto) so that it can be used to clean another endoscope (e.g., similar to the endoscope 10).

The shaft 28 may include a variety of forms. In some instances, the shaft 28 may be a relatively stiff rod. In other instances, the shaft 28 may be relatively flexible and capable of bending in one or more directions. The cleaning member/brush 30 may be secured to the shaft 28. In some instances, the cleaning member/brush 30 may be replaceable. In other words, the cleaning member/brush 30 may be removed from the shaft 28 and replaced by a new or different cleaning member/brush 30.

In some instances, the shaft 28 with the cleaning member/brush 30 coupled thereto can be used for a number of different cleaning procedures. In other instances, the shaft 28 with the cleaning member/brush 30 may be a single-use item, designed for a single cleaning procedure.

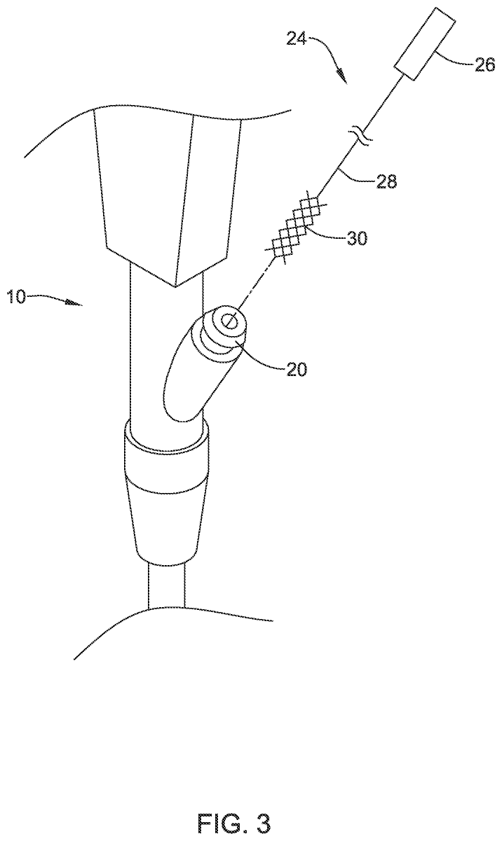

FIG. 3 schematically illustrates one example of use for the cleaning assembly 24, for example including the cleaning the working channel 18 of the endoscope 10. Briefly, the cleaning process may include inserting a part of the cleaning assembly 24 into a portion of the endoscope 10. For example, the cleaning member/brush 30 may be inserted into the biopsy port 20. The control unit 26 may be actuated in order to cause the cleaning assembly 24 (e.g., the shaft 28 and/or the cleaning member/brush 30) to move. When the endoscope 10 is considered to be sufficiently cleaned, the cleaning assembly 24 may be removed from the endoscope 10.

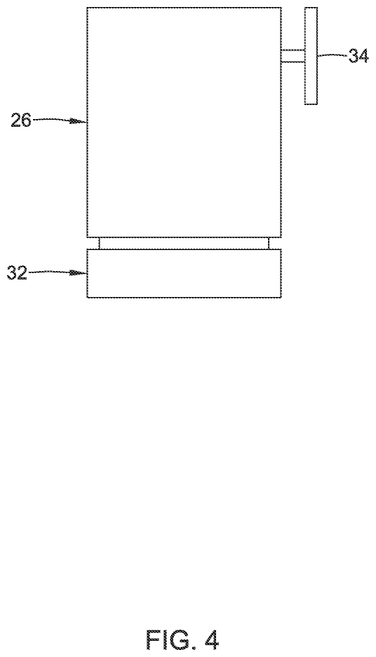

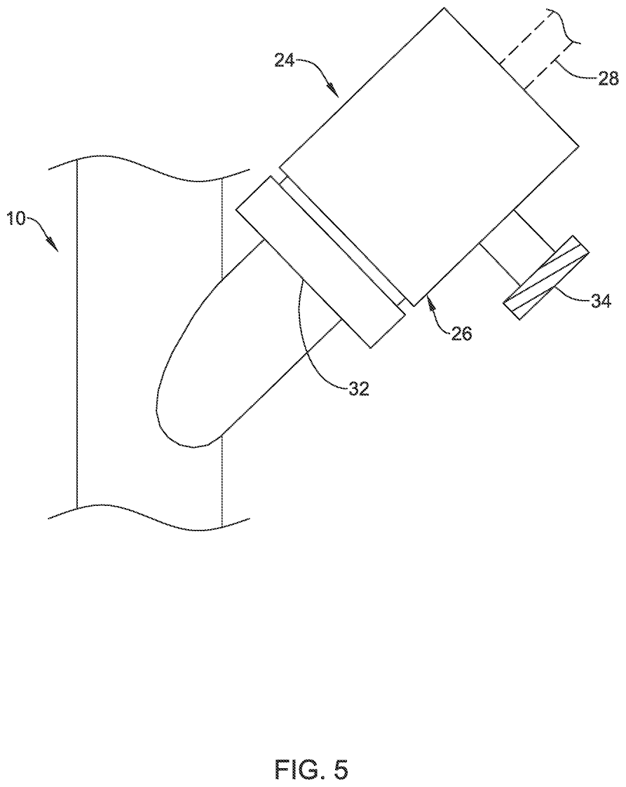

FIG. 4 illustrates the control unit 26. Here it can be seen that the control unit 26 may include a coupling region 32. In general, the coupling region 32 may be designed to engage and couple the control unit 26 to the biopsy port 20 of the endoscope 10. For example, the coupling region 32 may include a luer or other suitable connector. The attachment of the coupling region 32 to the biopsy port 20 is schematically depicted in FIG. 5. It can also be seen that the control unit 26 may also include a flush/wash port 34. The flush port 34 can used, for example, to flush the working channel 18 of the endoscope 10. In some instances, a fluid source may be coupled to the flush port 34 so that a fluid (e.g., a cleaning fluid) can be used in conjunction with the cleaning assembly 24. In some instances, the flush port 34 may include a valve for controlling the flow of fluid into (and/or out from) the flush port 34, as desired.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An apparatus for cleaning an endoscope, the apparatus comprising:
a control unit;
a shaft configured to be coupled to the control unit, the shaft having a distal end region;
a cleaning member coupled to the distal end region of the shaft; and
wherein the control unit is configured to move the cleaning member.

2. The apparatus of claim 1, wherein the control unit includes a motor.

3. The apparatus of claim 2, wherein the motor is a direct current motor.

4. The apparatus of claim 1, wherein the control unit is configured to rotate the cleaning member.

5. The apparatus of claim 1, wherein the control unit is configured to translate the cleaning member.

6. The apparatus of claim 1, wherein the control unit is configured to reciprocate the cleaning member.

7. The apparatus of claim 1, wherein the shaft includes a catheter shaft.

8. The apparatus of claim 1, wherein the cleaning member includes one or more bristles.

9. The apparatus of claim 1, wherein the control unit includes a coupling region configured to be coupled to a biopsy port of the endoscope.

10. The apparatus of claim 1, wherein the control unit includes a flush port.

11. An apparatus for cleaning an endoscope, the apparatus comprising:
an oscillating motor;
a single-use brush assembly coupled to the oscillating motor, the single-use brush assembly including a shaft having a brush coupled thereto; and
wherein the oscillating motor is configured to move the single-use brush assembly such that the single-use brush assembly can clean a channel of the endoscope.

12. The apparatus of claim 11, wherein the oscillating motor includes a direct current power supply.

13. The apparatus of claim 11, wherein the oscillating motor is configured to rotate the single-use brush assembly.

14. The apparatus of claim 11, wherein the oscillating motor is configured to translate the single-use brush assembly.

15. The apparatus of claim 11, wherein the oscillating motor is configured to reciprocate the single-use brush assembly.

16. The apparatus of claim 11, wherein the oscillating motor includes housing having a coupling member for coupling the oscillating motor to a biopsy port.

17. The apparatus of claim 11, wherein the oscillating motor includes housing having a flush port.

18. A method for cleaning a channel of an endoscope, the method comprising:
coupling a cleaning apparatus to a biopsy port, the cleaning apparatus including a control unit and a brush assembly coupled to the control unit;
wherein the brush assembly includes a shaft and a brush coupled to the shaft;
disposing the brush with the channel of the endoscope; and
actuating the control unit to cause the brush to move within the channel.

19. The method of claim 18, wherein the control unit includes a coupling member and wherein coupling a cleaning apparatus to a biopsy port includes securing the coupling member to the biopsy port.

20. The method of claim 18, wherein actuating the control unit to cause the brush to move within the channel includes translating the brush within the channel, rotating the brush within the channel, or both.
